(19) United States
(12) Patent Application Publication
(10) Pub. No.: US 2009/0143855 A1
(43) Pub. Date: Jun. 4, 2009

(54) Medical Device Including Drug-Loaded Fibers

(75) Inventors: Jan Weber, Maastricht (NL); Jaydeep Y. Kokate, Maple Grove, MN (US); Arif Iftekar, Santa Rosa, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 11/946,946

(22) Filed: Nov. 29, 2007

(51) Int. Cl. A61F 2/82

(52) U.S. Cl. 623/1.42

(57) ABSTRACT

An endovascular or intraluminal stent comprising an expandable framework including a plurality of interconnected undulating or otherwise connected segments, and a plurality of fibers disposed on the expandable framework. At least a portion of the plurality of fibers is loaded with a therapeutic agent.

### TECHNICAL FIELD

The present disclosure generally relates to medical devices including drug-loaded fibers placed therewith. More specifically, the disclosure pertains to prostheses, such as prosthetic grafts and endovascular stents incorporating drug-loaded fibers.

### BACKGROUND

Implantable medical devices, such as prosthetic grafts or endovascular stents, are used frequently in medical procedures. For instance, endovascular stents have been found useful in the treatment and repair of blood vessels after a stenosis has been treated by percutaneous transluminal coronary angioplasty (PTCA), percutaneous transluminal angioplasty (PTA), or other medical procedure in which the patency and/or integrity of a vessel lumen is improved. Stents may also be used to provide patency/integrity of a vessel lumen across a stenosis in cases in which no initial PTCA or, PTA procedure is performed. Stents have also garnered beneficial results in other applications. For instance, stents may also be implanted in other body lumens or vessels, such as the urethra, esophagus, bile duct, or the like in order to improve the patency/integrity of the body lumen and/or vessel.

During some medical procedures it may be advantageous to provide a therapeutic agent, such as a pharmacological substance or drug, at the location in which the stent is positioned during placement of the stent. Stents incorporating a pharmacological substance have been devised for this purpose. Drug-releasing stent devices have shown great potential in treating coronary artery disease, as well as in other treatment situations. As the use of drug-releasing stent devices becomes more frequent, there is an ongoing desire to provide improved techniques involving the incorporation and/or release of a therapeutic agent for delivery with an endovascular stent.

### SUMMARY

The disclosure is directed to prostheses, such as prosthetic grafts and endovascular stents incorporating drug-loaded fibers.

Accordingly, one illustrative embodiment is an endovascular stent comprising an expandable framework including a plurality of interconnected undulating or otherwise patterned segments, and a plurality of fibers disposed on the expandable framework. Each of the plurality of fibers includes an annular porous sidewall defining a central lumen which is at least in part loaded with a therapeutic agent.

Another illustrative embodiment is an endovascular stent comprising an expandable framework including a plurality of interconnected undulating or otherwise patterned segments, and a plurality of nanoporous ceramic fibers disposed on the expandable framework. At least a portion of the plurality of nanoporous ceramic fibers is loaded with a therapeutic agent.

Another illustrative embodiment is a method of forming a drug releasing medical device. Initially, a plurality of fibers, each having a generally porous annular sidewall over at least a portion of its length defining a central lumen extending through the fiber, are formed. The central lumen of each of the fibers may then be loaded with a therapeutic agent, and the plurality of fibers may be placed on a medical device.

Yet another illustrative embodiment is a method of treating a stenosis of a lumen of a patient. A stent comprising an expandable framework including a plurality of interconnected undulating or otherwise patterned segments, wherein a plurality of nanoporous ceramic fibers at least in part loaded with a therapeutic agent are disposed on the expandable framework may be provided. The stent including the plurality of nanoporous ceramic fibers loaded with the therapeutic agent may be placed across a stenosis of a lumen, and then the stent may be expanded to engage with the tissue wall of the stenosis. Once placed at the stenosis, the therapeutic agent may permeate or diffuse from the plurality of nanoporous ceramic fibers over a duration of time.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is an illustrative embodiment of an exemplary stent;

FIG. 2A is an enlarged view of a portion of the stent of FIG. 1 incorporating an arrangement of a plurality of drug-releasing fibers;

FIG. 2B is an enlarged view of a portion of the stent of FIG. 1 incorporating an alternative arrangement of a plurality of drug-releasing fibers;

FIG. 2C is an enlarged view of a portion of the stent of FIG. 1 incorporating an alternative arrangement of a plurality of drug-releasing fibers;

FIG. 3 is a schematic cross-section of an illustrative porous fiber;

FIG. 4 illustrates an exemplary electrospinning apparatus; and

FIG. 5 is an illustrative embodiment of a stent placement system including a stent incorporating a plurality of drug-releasing fibers.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

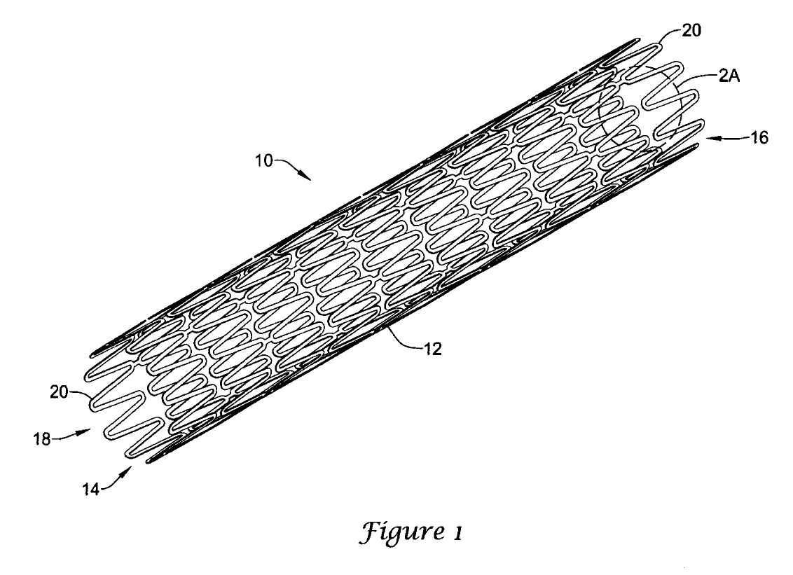

An exemplary implantable medical device, such as a prosthetic graft or endovascular stent incorporating drug-loaded fibers will now be described in more detail. An exemplary implantable medical device, illustrated as an endovascular stent 10, is shown in FIG. 1. Although illustrated as a stent, the implantable medical device may be any of a number of devices that may be introduced subcutaneously, percutaneously or surgically to be positioned within an organ, tissue, or lumen, such as a heart, artery, vein, urethra, esophagus, bile duct, or the like. The stent 10 may be any desired stent, such as an expandable (e.g., self-expandable or mechanically expandable) stent used during a percutaneous transluminal coronary balloon angioplasty (PTCA) or percutaneous transluminal angioplasty (PTA) procedure, for example. Some exemplary stents are disclosed in U.S. Pat. Nos. 6,730,117; 6,776,793; 6,945,993 and 6,981,986, which are each incorporated herein by reference.

The stent 10 may be a generally tubular member having a mesh framework 12 extending between a first end 14 and a second end 16, with a lumen 18 extending therethrough. The mesh framework 12 may include a plurality of interconnected undulating or otherwise patterned segments 20 defining interstitial spaces or openings therebetween. The stent 10 may be expandable from a collapsed configuration to an expanded configuration, either independently or by the application of mechanical force. The plurality of undulating or otherwise patterned segments 20 may be sufficiently flexible in order to be expandable once properly placed at the target site of interest.

The stent 10 may be formed of any desired material, such as a biocompatible material including biostable, bioabsorbable, biodegradable or bioerodible materials. For instance, the stent 10 may be formed of a metallic material or a polymeric material. Some suitable metallic materials include, but are not necessarily limited to, stainless steel, tantalum, tungsten, nickel-titanium alloys such as those possessing shape memory properties commonly referred to as nitinol, nickel-chromium alloys, nickel-chromium-iron alloys, cobalt-chromium-nickel alloys, or other suitable metals, or combinations or alloys thereof. Some suitable polymeric materials include, but are not necessarily limited to, polyamide, polyether block amide, polyethylene, polyethylene terephthalate, polypropylene, polyvinylchloride, polyurethane, polytetrafluoroethylene, polysulfone, and copolymers, blends, mixtures or combinations thereof.

The stent 10 may be covered or incorporated with a plurality of fibers 50, such as nanofibers or microfibers, in any appropriate fashion. (The fibers 50 are not illustrated in FIG. 1 for the sake of clarity). The fibers 50 may be placed on, interwoven with, wrapped around, or otherwise incorporated with the stent 10 in any desired fashion. The plurality of fibers 50 covering or incorporated with the stent 10 are intended to be distinguishable from a coating or laminated layer placed on and conforming to the outer surface of the stent 10. For example, the plurality of fibers 50 may be randomly oriented about the outer surface of the stent 10 leaving portions of the outer surface of the expandable framework 12 exposed and visible through the random arrangement of fibers 50. In some embodiments, the plurality of fibers 50 are nonconforming with the outer surface and/or the inner surface of the expandable framework 12. Thus in some embodiments, the plurality of fibers 50 may be a three-dimensional fibrous construct having various spaces between adjacent fibers 50 loosely blanketing the expandable framework 12 of the stent 10. Within the fibrous construct, a discrete fiber 50 may be readily discernible from an adjacent fiber 50.

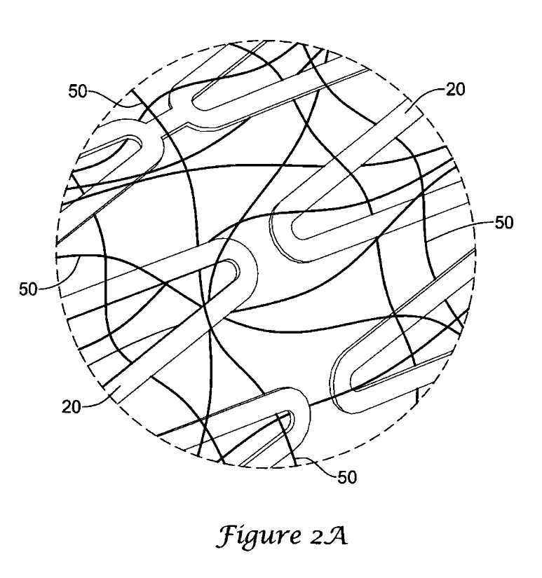

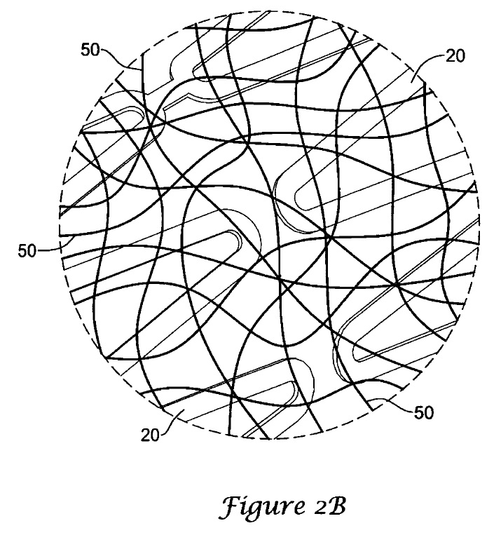

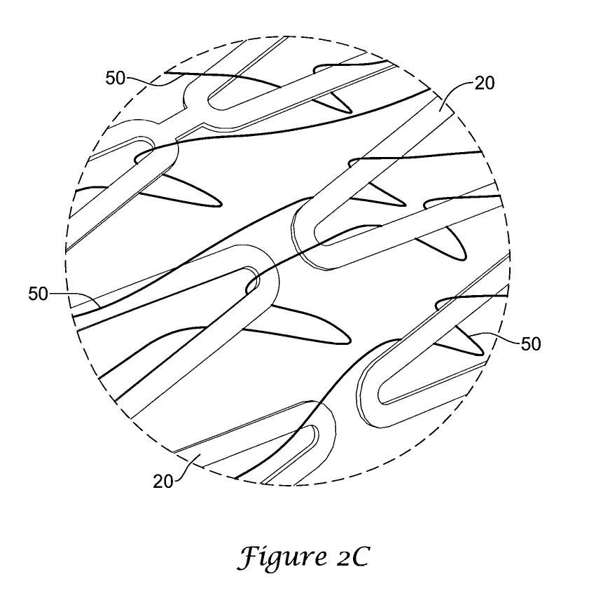

For instance, as shown in FIG. 2A, which is an expanded view of a portion of the stent 10 incorporating a plurality of fibers 50, the fibers 50 may be interwoven or entangled with the undulating or otherwise patterned segments 20 of the stent 10. In such an instance, a portion of the fibers 50 may extend over the exterior of the undulating segments 20 while a portion of the fibers 50 may extend through openings of the stent 10 to a location radially interior to the undulating segments 20, leaving a portion of the outer surface and/or inner surface of the framework 12 of the stent 10 exposed and accessible to tissue and/or blood while the stent 10 is in a collapsed state and/or in an expanded state. In some embodiments, the outer surface of the expandable framework 12 of the stent 10 may be visible through the mat of fibers 50 when the stent 10 is retained in a collapsed state as well as when the stent 10 is in an expanded state. As shown in FIG. 2A, in some embodiments, the outer surface of the expandable framework 12 may be exposed throughout the entanglement of fibers 50.

In an alternative configuration as shown in FIG. 2B, the fibers 50 may be wrapped around the stent 10. In such an instance, the plurality of fibers 50 may be a woven, non-woven or entangled mat of fibers 50 placed over the outer surface of the stent 10. As shown in FIG. 2B, the outer surface of the expandable framework 12 may be exposed through the mat of fibers 50. Thus, the outer surface of the expandable framework 12 of the stent 10 may be visible through the mat of fibers 50 when the stent 10 is retained in a collapsed state as well as when the stent 10 is in an expanded state, leaving a portion of the outer surface and/or inner surface of the framework 12 of the stent 10 exposed and accessible to tissue and/or blood while the stent 10 is in a collapsed state and/or in an expanded state.

Another configuration of fibers 50 incorporated with the stent 10 is shown in FIG. 2C. In some embodiments, such as shown in FIG. 2C, a single fiber 50 may extend into the interior of the stent 10 through an interstitial space between adjacent undulating segments 20 of the framework 12 of the stent 10 and extend back out to the exterior of the stent 10 through the same interstitial space between adjacent undulating segments 20 of the framework 12 of the stent 10. Additional fibers 50 may likewise both extend into and extend back out of a single interstitial space between adjacent undulating segments 20 of the framework 12 of the stent 10. In some embodiments, fibers 50 may be placed on the outer surface of the stent 10. As shown in FIG. 2C, the outer surface of the expandable framework 12 in some embodiments may be exposed through the mat of fibers 50. Once the fibers 50 are placed on the outer surface of the stent 10, a portion of a fiber 50 may be pushed inward through an interstitial space between two adjacent undulating segments 20 of the framework 12 so that the fiber 50 extends radially inward of the inner surface of the expandable framework 12 of the stent 10. Additional fibers 50 may likewise be pushed inward through an interstitial space between two adjacent undulating segments 20 of the framework 12 so that these additional fibers 50 extend radially inward of the inner surface of the expandable framework 12 of the stent 10. After one or more of the fibers 50 have been pushed radially inward through interstitial spaces of the framework 12, the fiber or fibers 50 may be pushed slightly axially within the stent 10 so that the doubled-over portion (i.e., the portion of the fiber 50 extending into the lumen 18 of the stent 10) of a fiber 50 may be pushed axially underneath an undulating segment 20. It can be seen that pushing the fiber 50 slightly axially will cause the doubled-over portion of the fiber 50 within the lumen 18 of the stent 10 to hook under an undulating segment 20 of the stent 10 to secure the fiber 50 to the stent 10. Performing such a technique with a plurality of fibers 50 of a stent 10 will result in the fibers 50 being entangled with the expandable framework 12 of the stent 10. The fibers 50 may be pushed by any desired means. For example, in some embodiments, manipulation of the fibers 50 may be performed by short burst of air, with a brush, or other tool.

Within the materials science industry, fibers with diameters below about 500 nanometers, and typically between about 100 nanometers to about 500 nanometers, are generally classified as nanofibers. In some embodiments the fibers 50 may be nanofibers, having a diameter of less than about 500 nanometers. For instance, in some embodiments, the diameter of the fibers 50 may be between about 100 nanometers to about 500 nanometers. However, in other embodiments, the fibers 50 may have an outer diameter greater than 500 nanometers. For instance, in some embodiments the fibers 50 may have an outer diameter of about 0.5 micrometers to about 5.0 micrometers, about 0.5 micrometers to about 2.0 micrometers, or about 0.5 micrometers to about 1.0 micrometers.

The fibers 50 may be formed from a variety of materials, such as biostable or bioabsorbable materials. Some suitable materials may include metals, ceramics or polymers, for example. For instance, in some embodiments the fibers 50 may be ceramic fibers, such as metal oxide fibers. Some suitable examples of metal oxide ceramic fibers include aluminum oxide, copper oxide, chromium oxide, magnesium oxide, niobium oxide, tantalum oxide, tantalum-niobium oxide, titanium oxide, vanadium oxide, vanadium-titanium oxide, combinations, mixtures or blends thereof, or the like. Some suitable examples of polymeric fibers include polyurethane, polyvinyl alcohol, poly(lactic glycolic) acid, polyethylene, polyethylene oxide, polyethylene terephthalate, or polyester, or mixtures, combinations, blends or co-polymers thereof, or the like.

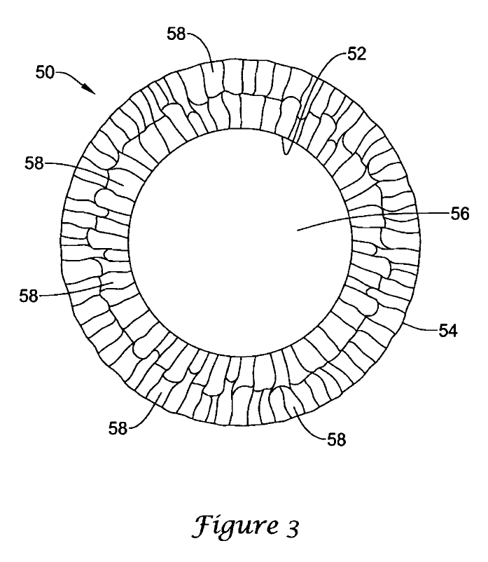

As shown in FIG. 3, the fibers 50 may be elongate hollow tubular fibers, having determinable inner wall diameter and outer wall diameter sizes. The fibers 50 may include an annular sidewall having an inner surface 52 and an outer surface 54. The inner surface 52 of the annular sidewall of the fibers 50 may define an inner central lumen 56 extending coaxially along the longitudinal length of the fibers 50. In some embodiments, the fibers 50 may have an inner diameter of about 10 nanometers to about 3 micrometers, about 50 nanometers to about 2 micrometers, about 100 nanometers to about 1 micrometer, or about 50 nanometers, about 100 nanometers, about 200 nanometers, about 300 nanometers, about 400 nanometers, about 500 nanometers, about 1 micrometer, about 2 micrometers, or about 3 micrometers, for example.

As shown in FIG. 3, the annular sidewall of the fibers 50 may be porous, thereby allowing certain substances to permeate or diffuse through the sidewall of the fibers 50 through the pores or interstitial spaces 58. The sidewall may have any desired porosity. For example, typically the porous sidewall of the fiber 50, which may be a nanoporous sidewall in some instances, may have an average pore size of about 1 nanometer to about 1,000 nanometers. The IUPAC Compendium of Chemical Terminology has presented a standard for the classification of nanoporous bodies. In view of the IUPAC classification, nanoporous bodies are divided into three classes, microporous bodies having a pore size of less than 2 nanometers, mesoporous bodies having a pore size of between 2 nanometers to 50 nanometers, and macroporous bodies having a pore size of over 50 nanometers. Thus, the sidewall of the fiber 50 may have an average pore size of less than about 2 nanometers, between about 2 nanometers to about 50 nanometers, or greater than about 50 nanometers, for example. The porosity (e.g., the percentage of interstitial volume to total volume) of the fibers 50 may be about 10% or more, about 20% or more, about 30% or more, about 40% or more, about 50% or more, about 60% or more, about 70% or more, or about 80% or more, for example.

The fibers 50 may be loaded with a therapeutic agent. For instance, the central lumen 56 of the fibers 50 may be filled with a therapeutic agent. For example, a therapeutic agent may be flushed through the central lumen 56 of the fibers 50, or a therapeutic agent may be drawn into the central lumen 56 of the fibers 50 by capillary action. As the inner diameter and length of the fiber 50 may be precisely controlled, the internal volume of the fibers 50 may be known, and thus the precise volume of the therapeutic agent loaded into the fibers 50 may be accurately determined. A desired quantity of fibers 50 of known size having a therapeutic agent loaded therewith may be incorporated with the stent 10. Thus, precise quantities of a therapeutic agent may be included with the stent 10. Once implanted in a body, the therapeutic agent may diffuse through the porous sidewall of the fibers 50 over a predetermined period of time dictated, at least in part, by the average pore size of the porous sidewall of the fibers 50. Thus, the rate of release of the therapeutic agent may be known and dictated, at least in part, by the porosity of the fibers 50. For instance, the porosity of the fibers 50 may be chosen to controllably release the therapeutic agent over a period of minutes, hours, days, weeks, months, years, etc. In some embodiments, the duration of release of the therapeutic agent from the fibers 50 may be about 1 hour, about 2 hours, about 3 hours, about 4 hours, about 5 hours, about 6 hours, about 12 hours, about 1 day, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 1 year, about 2 years, or longer. In some embodiments the duration for controlled release of the therapeutic agent may be about 1 hour to about 24 months. Thus, fibers 50 may be chosen for their porosity such that a desired rate of drug release is provided.

The therapeutic agent may be any medicinal agent which may provide a desired effect. Suitable therapeutic agents include drugs, genetic materials, and biological materials. For instance, in some embodiments, the therapeutic agent may include a drug which may be used in the treatment of restenosis. Some suitable therapeutic agents which may be loaded in the fibers 50 include, but are not necessarily limited to, antibiotics, antimicrobials, antiproliferatives, antineoplastics, antioxidants, endothelial cell growth factors, thrombin inhibitors, immunosuppressants, anti-platelet aggregation agents, collagen synthesis inhibitors, therapeutic antibodies, nitric oxide donors, antisense oligonucleotides, wound healing agents, therapeutic gene transfer constructs, peptides, proteins, extracellular matrix components, vasodialators, thrombolytics, anti-metabolites, growth factor agonists, antimitotics, steroidal and non-steroidal anti-inflammatory agents, angiotensin converting enzyme (ACE) inhibitors, free radical scavengers, and anticancer chemotherapeutic agents.

In certain embodiments, the therapeutic agent is useful for inhibiting cell proliferation, contraction, migration, hyperactivity, or addressing other conditions. The term “therapeutic agent” encompasses drugs, genetic materials, and biological materials. Non-limiting examples of suitable therapeutic agents include heparin, heparin derivatives, urokinase, dextrophenylalanine proline arginine chloromethylketone (PPack), enoxaprin, angiopeptin, hirudin, acetylsalicylic acid, tacrolimus, everolimus, rapamycin (sirolimus), amlodipine, doxazosin, glucocorticoids, betamethasone, dexamethasone, prednisolone, corticosterone, budesonide, sulfasalazine, rosiglitazone, mycophenolic acid, mesalamine, paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, lidocaine, bupivacaine, ropivacaine, D-Phe-Pro-Arg chloromethyl ketone, platelet receptor antagonists, anti thrombin antibodies, anti platelet receptor antibodies, aspirin, dipyridamole, protamine, hirudin, prostaglandin inhibitors, platelet inhibitors, trapidil, liprostin, tick antiplatelet peptides, 5-azacytidine, vascular endothelial growth factors, growth factor receptors, transcriptional activators, translational promoters, antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin, cholesterol lowering agents, vasodilating agents, agents which interfere with endogenous vasoactive mechanisms, antioxidants, probucol, antibiotic agents, penicillin, cefoxitin, oxacillin, tobranycin, angiogenic substances, fibroblast growth factors, estrogen, estradiol (E2), estriol (E3), 17-beta estradiol, digoxin, beta blockers, captopril, enalopril, statins, steroids, vitamins, taxol, paclitaxel, 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, 2′-O-ester with N-(dimethylaminoethyl) glutamide hydrochloride salt, nitroglycerin, nitrous oxides, nitric oxides, antibiotics, aspirins, digitalis, estrogen, estradiol and glycosides. In one embodiment, the therapeutic agent is taxol (e.g., Taxol®), or its analogs or derivatives. In another embodiment, the therapeutic agent is paclitaxel. In yet another embodiment, the therapeutic agent is an antibiotic such as erythromycin, amphotericin, rapamycin, adriamycin, etc.

The term “genetic materials” means DNA or RNA, including, without limitation, DNA/RNA encoding of a useful protein stated below, intended to be inserted into a human body including viral vectors and non-viral vectors.

The term “biological materials” include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other non-genetic therapeutic agents include:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, amlodipine and doxazosin;
anti-inflammatory agents such as glucocorticoids, betamethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, taxol and its analogs or derivatives;
anesthetic agents such as lidocaine, bupivacaine, and ropivacaine;
anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents; vasodilating agents; and agents which interfere with endogenous vasoactive mechanisms;
anti-oxidants, such as probucol;
antibiotic agents, such as penicillin, cefoxitin, oxacillin, tobranycin, macrolides such as rapamycin (sirolimus) and everolimus;
angiogenic substances, such as acidic and basic fibroblast growth factors, estrogen including estradiol (E2), estriol (E3) and 17-beta estradiol; and
drugs for heart failure, such as digoxin, beta-blockers, angiotensin-converting enzyme (ACE) inhibitors including captopril and enalopril, statins and related compounds. Preferred biologically active materials include anti-proliferative drugs such as steroids, vitamins, and restenosis-inhibiting agents. Preferred restenosis-inhibiting agents include microtubule stabilizing agents such as Taxol®, paclitaxel (i.e., paclitaxel, paclitaxel analogues, or paclitaxel derivatives, and mixtures thereof). For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt.

Other preferred therapeutic agents include nitroglycerin, nitrous oxides, nitric oxides, antibiotics, aspirins, digitalis, estrogen derivatives such as estradiol and glycosides.

In certain embodiments, the therapeutic agents for use in the medical devices of the present disclosure can be synthesized by methods well known to one skilled in the art. Alternatively, the therapeutic agents can be purchased from chemical and pharmaceutical companies.

In some embodiments, the central lumen 56 of the fibers 50 may be loaded with a mixture of a therapeutic agent and a polymer carrier. Thus elution of the therapeutic agent may be controlled, at least in part, by the degeneration and/or drug releasing properties of the polymer carrier.

The therapeutic agent may be contained in the central lumen 56 of the fibers 50 by closing or sealing the open ends of the fibers 50 once the therapeutic agent has been loaded in the fibers 50. For example, in some embodiments, the ends of the fibers 50 may be sealed by dipping the fibers 50 into a slowly dissolving biomaterial, a polymer or a metal. In other embodiments, an adhesive may be used to seal the ends of the central lumen 56 of the fibers 50.

In other embodiments, the fibers 50 may be non-hollow, thus not including a central lumen loaded with a therapeutic agent. Instead, a therapeutic agent may be loaded in the nanoporosity of the fibers 50. In other words, a therapeutic agent may be loaded in the interstitial spaces 58 of the fibers 50. In such an instance, the quantity of therapeutic agent included with the fiber 50 may be dictated by the porosity of the fibers 50. In other words, fibers 50 with larger and/or higher quantities of pores would be able to be loaded with a greater content of a therapeutic agent.

The therapeutic agent may be locally released from the fiber 50 in a controlled, time-released manner. For instance, the therapeutic agent may be released through the interstitial spaces of the sidewall of the fiber 50 over a determined period of time. For instance, the therapeutic agent may be released from the fiber 50 over a period of minutes, hours, days, weeks, months, years, etc. In some embodiments, the duration of release of the therapeutic agent from the fibers 50 may be about 1 hour, about 2 hours, about 3 hours, about 4 hours, about 5 hours, about 6 hours, about 12 hours, about 1 day, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 1 year, about 2 years, or longer. Thus, the porosity of the sidewall of the fiber 50 may control the rate of permeation of the therapeutic agent from the fiber 50. For instance a fiber 50 having a relatively more porous (e.g., larger average pore size) sidewall may diffuse the therapeutic agent at a higher rate than a fiber 50 having a relatively less porous (e.g., smaller average pore size) sidewall.

Electrospinning is one possible technique for producing fibers, such as nanofibers and/or microfibers, having cylindrical-like geometries. However, other processes, such as molding, electrospraying, extrusion and the like, may be utilized to form fibers. Electrospinning, generally speaking, is a process of spinning fibers with the help of electrostatic forces. Electrospinning has been found to be an advantageous process due at least in part to the ability to maintain consistency in producing fibers. Additionally, electrospinning has been found to result in the formation of fibers having a relatively small pore size and relatively high surface area.

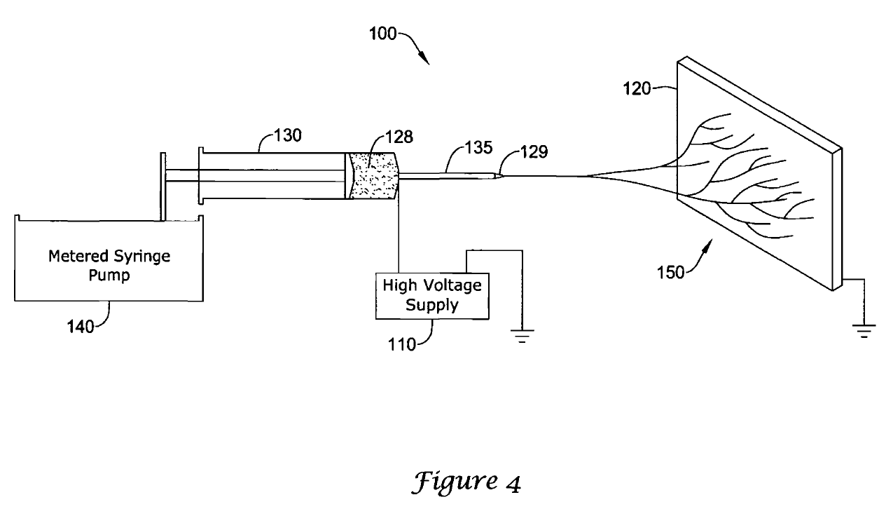

FIG. 4 schematically illustrates a typical apparatus used for electrospinning fibers, such as nanofibers and/or microfibers. The electrospinning apparatus 100 includes a high voltage electric source 110, a collector plate 120 and a syringe 130 including a needle 135, or other nozzle connected to a syringe pump 140 for precisely metering the flow rate of the syringe 130. The high voltage electric source 110 typically creates a voltage between about 10 kV to about 50 kV, although other voltages may be found effective in certain applications. The high voltage electric source 110, which may have a positive or negative polarity, creates an electric field between a droplet of fluid at the tip of the needle 135 of the syringe 130 and the collector plate 120. The collector plate 120 may be any desired shape. For example, the collector plate 120 may be a flat plate, a rotating drum, a rotating disc having a sharpened edge, or the like. Additionally, the collector plate 120 may include any desired conductive material. For example, the collector plate 120 may be aluminum, copper, or other material as desired.

The syringe 130 including the needle 135, or other nozzle, is spaced a predetermined distance from the collector plate 120. For instance, in some embodiments the needle 135 may be placed about 10 centimeters to about 25 centimeters from the collector plate 120, or at another distance as desired. The syringe 130 is attached to a syringe pump 140, which provides a flow of a liquid mixture 128 to the needle 135 of the syringe 130. The liquid mixture 128 may be a solution, a suspension, a gel, a sol, or other precursor substance for forming the fibers 150. The liquid mixture 128 may include a precursor substance for forming the fibers 150 as well as a carrier, for example a solvent such as ethanol, propanol, or acetone.

One electrode of the high voltage electric source 110 is placed in electrical contact with the liquid mixture 128 while another electrode is connected to the collector plate 120, creating an electrostatic force therebetween. As the voltage is increased, an electrostatic force builds up on the drop of liquid mixture 128 at the tip of the needle 135. This force, which acts in a direction opposing the surface tension of the drop, causes the drop of fluid to elongate, forming a conical shape known as a Taylor cone 129. When the electrostatic force overcomes the surface tension of the drop, a charged, continuous jet of fluid is discharged from the cone and accelerates toward the collector plate 120 with a whipping motion. As the fluid travels toward the collector plate 120, the jet thins and dries, creating a nonwoven mat of randomly oriented fibers 150 on the collector plate 120.

It is noted that in some embodiments the electrospinning apparatus 100 may deviate from that illustrated in FIG. 4. For example, in some embodiments, the collector plate 120 may be substituted for a pair of conductive strips separated by a gap, the polarity of the power supply may be reversed, the apparatus 100 may be oriented in a vertical orientation, or the like.

Factors which may influence the electrospinning process include, among other parameters, the magnitude of the applied electrical potential, the distance between the needle 135 and the collector plate 120, and characteristics of the liquid mixture 128 such as the viscosity, concentration, conductivity, surface tension and/or flow rate of the liquid mixture 128, as well as environmental conditions, among others. For example, adjusting the distance between the needle 135 and the collector plate 120 and/or the applied voltage may result in a change in the characteristics of the fibers 150. A decrease in the distance between the needle 135 and the collector plate 120 may result in a decrease in beading of the fibers 150, whereas an increase in the distance between the needle 135 and the collector plate 120 may result in an increase in beading of the fibers 150. Furthermore, increasing the distance between the needle 135 and the collector plate 120 may decrease the outer diameter of the fibers 150, whereas decreasing the distance between the needle 135 and the collector plate 120 may increase the outer diameter of the fibers 150. Additionally, decreasing the voltage may result in an increase in beading of the fibers 150, whereas an increase in the voltage may result in a decrease in beading of the fibers 150. Also, it has been found that the fiber diameter and/or pore size may increase with an increase in the flow rate of the liquid mixture 128 from the syringe 130.

In some embodiments, the fibers 150 may subsequently be subjected to a calcination process or other process. For example, in some embodiments, after the fibers 150 are formed in the electrospinning process, the fibers 150 may be subjected to a calcination temperature of about 400° C., about 500° C., about 600° C., about 700° C., about 800° C., about 900° C., or about 1000° C. However, higher or lower temperatures may be desired in some instances. Such a process may be found to further influence the morphology and crystallinity of the fibers 150. For example, calcination and/or solvent extraction may be used to remove organic components from the formed fibers 150.

Subsequent to formation of the fibers 150, the fibers 150 may be loaded or filled with a therapeutic agent. In some embodiments the fibers 50 may include a therapeutically effective amount of one or more therapeutic agents for inhibiting cell proliferation, contraction, migration or hyperactivity, inflammation, thrombosis, restenosis, or the like. For instance, in some embodiments a therapeutic agent may be disposed in the central lumen of the fibers 150, and/or a therapeutic agent may be disposed in the interstitial spaces of the fibers 150. In some embodiments, the therapeutic agent may be flushed through the central lumen of the fibers 150, or the therapeutic agent may be drawn into the central lumen of the fibers 150 through capillary action. In other embodiments, the fibers 150 may be submerged in or sprayed with a therapeutic agent or a solution including a therapeutic agent. The fibers 150 may then be incorporated with an implantable medical device such as the stent 10 illustrated in FIG. 1 or any other desired medical device in which controlled, drug-releasing capabilities are desired. For instance, the fibers 150 may be interwoven with, entwined with, entangled with, wrapped around, or otherwise incorporated with the stent 10. The fibers 150 may be incorporated with the stent 10 prior to or subsequent positioning the stent 10 on a catheter balloon or other delivery/deployment device.

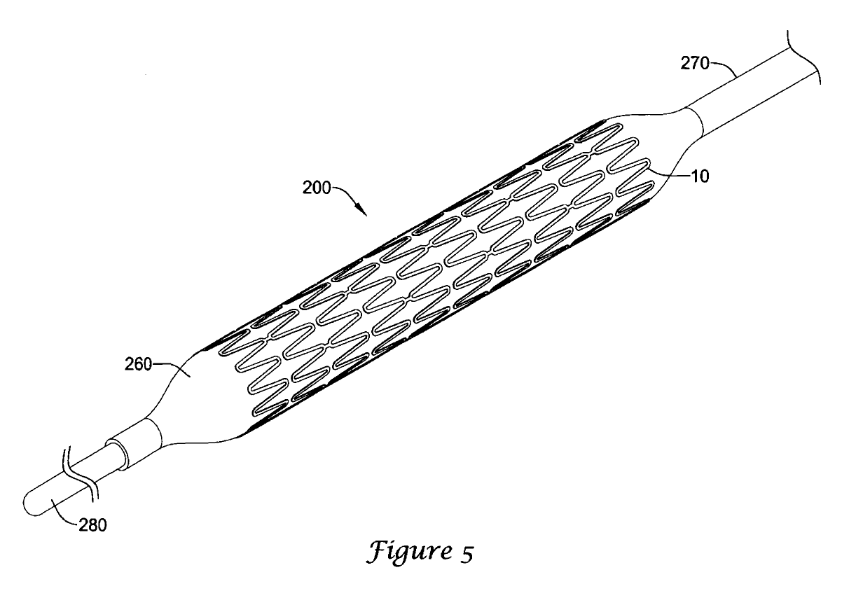

FIG. 5 illustrates an exemplary stent placement assembly 200 including a stent 10 incorporating the drug-releasing fibers 50 as described herein. (The fibers 50 are not illustrated in FIG. 5 for the sake of clarity). The assembly 200 includes an inflatable balloon 260 secured to a catheter shaft 270. The stent 10 may be positioned over the inflatable balloon 260. For example, the stent 10 may be crimped, or otherwise compressed over the inflatable balloon 260. A plurality of fibers 50 may be incorporated with the stent 10. For example, in some embodiments, the fibers 50 may be incorporated with the stent 10 prior to securing the stent 10 over the balloon 260. For instance, in some embodiments the fibers 50 may be interwoven and/or entangled with the undulating segments 20 of the stent 10. However, in other embodiments, the fibers 50 may be placed on the stent 10 subsequent to securing the stent 10 over the balloon 260. For instance, in some embodiments, the fibers 50 may be loosely wound around the stent 10 after the stent 10 is crimped onto the balloon 260.

During a medical procedure, a guidewire 280 may be advanced through a lumen, such as a blood vessel, of a patient to a remote location, such as distal a stenosis. The stent placement assembly 200 may be advanced over the guidewire 280 such that the balloon 260 and/or the stent 10 is positioned proximate the stenosis. The stent 10 may be expanded to engage the tissue surface of the stenosis. For example, the balloon 260 may be expanded in order to expand the stent 10 to contact the tissue of the vessel. Upon expansion of the stent 10, the fibers 50 may be interposed between the tissue surface and the stent 10. Subsequently, the catheter 270, including the balloon 260, may be withdrawn from the lumen, leaving the stent 10 in place at the stenosis.

In some embodiments, the fibers 50 may be incorporated with a biodegradable polymeric stent structure or a bioerodible metal stent structure, such as a magnesium or iron stent. In such an embodiment, the fibers 50 may serve multiple purposes. Initially, the fibers 50 may deliver a therapeutic agent to the surrounding tissue as the stent structure is degrading and/or eroding. The fibers 50 may also serve as a reinforcement structure for the stent structure such that as the stent structure degrades and/or erodes, the fibers 50 remain interconnected, providing continued support. It is also contemplated that the fibers 50 may be used as aneurism fill-material surrounding a covered stent structure.

In some embodiments, the inclusion of the fibers 50 with the expandable framework 12 of the stent 10 may promote tissue growth around the stent 10 once implanted in a vessel lumen. This may be due, at least in part, to the exposed surface area of the fibers 50 as a consequence of the porosity of the fibers 50. Thus, the porous fibers 50 may more readily promote tissue growth around the stent 10 than instances in which a stent is coated with a polymeric layer of material. Therefore, in some instances, in may be desirable to incorporate fibers 50 not loaded with a therapeutic agent and/or fibers 50 loaded with a therapeutic agent with a stent 10 in order to promote tissue growth around the stent 10.

There are numerous additional perceived advantages of the presently described nanoporous fibers. For instance, adhesion problems commonly encountered with stent coatings are eliminated. Additionally, application of the disclosed fibers to the stent does not adversely affect the morphology of the stent material, which may be the case when applying a coating directly to a stent surface.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A stent comprising:
an expandable framework having a first end, a second end, an outer surface, and an inner surface defining a lumen, the expandable framework including a plurality of interconnected segments; and
a plurality of fibers disposed on the expandable framework;
wherein at least a portion of the plurality of fibers include an annular porous sidewall having an outer diameter and an inner diameter, the inner diameter of the annular porous sidewall defining a central lumen;
wherein at least a portion of the central lumen of at least some of the plurality of fibers is loaded with a therapeutic agent.

2. The stent of claim 1, wherein the plurality of fibers are disposed on the outer surface of the expandable framework.

3. The stent of claim 1, wherein the plurality of fibers are interwoven with the expandable framework.

4. The stent of claim 1, wherein the plurality of fibers are wrapped around the outer surface of the expandable framework.

5. The stent of claim 1, wherein the plurality of the fibers have an average pore size of about 1 nanometer to about 1000 nanometers.

6. The stent of claim 1, wherein the plurality of fibers have an average pore size of less than about 2 nanometers.

7. The stent of claim 1, wherein the plurality of fibers have an average pore size of about 2 nanometers to about 50 nanometers.

8. The stent of claim 1, wherein the plurality of fibers have an average pore size greater than about 50 nanometers.

9. The stent of claim 1, wherein the porosity of the plurality of fibers allows diffusion of the therapeutic agent through the sidewall of the plurality of fibers.

10. An intraluminal stent for placement within a vessel lumen, the intraluminal stent comprising:
an expandable framework having a first end, a second end, an outer surface, and an inner surface defining a lumen, the expandable framework including a plurality of interconnected segments; and
a plurality of nanoporous ceramic fibers disposed on the expandable framework, wherein at least a portion of the plurality of nanoporous ceramic fibers is loaded with a therapeutic agent.

11. The intraluminal stent of claim 10, wherein the plurality of nanoporous ceramic fibers forms a nonwoven mesh.

12. The intraluminal stent of claim 10, wherein the plurality of nanoporous ceramic fibers comprise a metal oxide.

13. The intraluminal stent of claim 10, wherein the plurality of nanoporous ceramic fibers are interwoven with the expandable framework.

14. The intraluminal stent of claim 10, wherein the plurality of nanoporous ceramic fibers are wrapped around an outer surface of the expandable framework.

15. The intraluminal stent of claim 10, wherein each of the nanoporous ceramic fibers has a central lumen, wherein the therapeutic agent is loaded within the central lumen of the nanoporous ceramic fibers.

16. The intraluminal stent of claim 10, wherein each of the nanoporous ceramic fibers comprises a plurality of interstitial spaces, wherein the therapeutic agent is loaded within the interstitial spaces of the nanoporous ceramic fibers.

17. A method of forming a drug releasing medical device, the method comprising:
forming a plurality of fibers, each fiber having a porous annular sidewall having an outer surface and an inner surface, the inner surface of the fiber defining a central lumen extending through the fiber;
loading the central lumen of at least a portion of the fibers with a therapeutic agent; and
placing the plurality of fibers on a medical device.

18. The method of claim 17, wherein the plurality of fibers are formed through an electrospinning process.

19. The method of claim 17, wherein the medical device includes an expandable framework, wherein the plurality of fibers are interwoven with the expandable framework.

20. The method of claim 17, wherein the medical device includes an expandable framework having an outer surface, wherein the plurality of fibers are wrapped around the outer surface of the expandable framework.

21. The method of claim 17, wherein the plurality of fibers comprise ceramic fibers.

22. A method of treating a stenosis of a lumen of a patient, the method comprising:
providing a stent comprising an expandable framework having a first end, a second end, an outer surface, and an inner surface defining a lumen, the expandable framework including a plurality of interconnected segments, wherein a plurality of nanoporous ceramic fibers are disposed on the expandable framework, wherein each of the plurality of nanoporous ceramic fibers is loaded with a therapeutic agent;
placing the stent including the plurality of nanoporous ceramic fibers loaded with the therapeutic agent across a stenosis of a lumen;
expanding the stent to engage with a tissue wall of the stenosis; and
diffusing the therapeutic agent from the plurality of nanoporous ceramic fibers over a duration of time.

23. The method of claim 22, wherein the plurality of nanoporous ceramic fibers are interposed between the expandable framework and the tissue wall of the stenosis.

24. The method of claim 22, wherein the therapeutic agent is loaded in a central lumen of the nanoporous ceramic fibers.

25. The method of claim 22, wherein the therapeutic agent diffuses through a porous sidewall of the nanoporous ceramic fibers.
